(19) United States
(12) Patent Application Publication
(10) Pub. No.: US 2023/0044168 A1
(43) Pub. Date: Feb. 9, 2023

(54) AIR PURIFIER MASK AND METHOD THEREOF

(71) Applicant: Infosys Limited, Bangalore (IN)

(72) Inventors: Sundaresan Poovalingam, Bangalore (IN); Shashidhar CHAMARAJU, Mysore (IN); Anjan Kumar PANDA, Mysore (IN); Chethan NINGAPPA, Chikmagalur (IN); Rajesh SANGALGE, Bangalore (IN)

(21) Appl. No.: 17/510,868

(22) Filed: Oct. 26, 2021

(30) Foreign Application Priority Data
Aug. 4, 2021 (IN) 202041042514

(51) Int. Cl. A62B 7/10; A62B 9/00; A62B 18/02; A61L 9/16

(52) U.S. Cl. CPC A62B 7/10; A62B 9/003; A62B 18/02; A61L 9/16; A61L 2209/14; A61L 2209/15; A61L 2209/111

(57) ABSTRACT

An air purifying mask having a face mask along with components some of which are arranged inside a casing. The casing may contain an air filtering system, along with other associated components for the mask including blower, appropriate pressure, flow control and filtration settings, and heating control. A flexible attachable/detachable connecting coaxial duct for inhale and exhale, may connect the mask and the air filtration system. The system may have a heating element for self-cleaning and temperature control. The heating element may also help for disinfection of air path within the system and air mask.

This application claims the benefit of Indian Patent Application Ser. No. 202041042514 filed Aug. 4, 2021, which is hereby incorporated by reference in its entirety.

### FIELD

The present disclosure relates to an air purifying mask and method.

### BACKGROUND

The use of air masks while travelling through polluted or infected air is well known. Face masks are part of an infection control strategy to eliminate cross-contamination. When someone cough, talk, sneeze they could release germs into the air that may infect others nearby. However, for a first-time mask user, it takes few days to get used to breathing through a mask. For those with chronic respiratory condition such as asthma or COPD, covering the mouth and nose can be especially challenging. The physical barrier of the mask makes it harder to take in air. Many people with chronic lung conditions find it harder to breathe in hot, humid air. Mask traps some carbon dioxide during exhalation, thus ending up in breathing in stale air that is warmer and moist. Prolonged use may cause dizziness due to breathing in too much-exhaled CO₂, as mask traps CO₂. Normal physical activity can cause discomfort, loss of reflexes and conscious, leading to increased fatigue. After few uses of the mask, the masks become unusable. They get infected and are not fit for further use.

### SUMMARY

Provided is an air purifier mask which has a face mask, a casing which is connected to the face mask and the casing has an inlet filter for filtering incoming air, pressure sensors and regulators for monitoring and regulating a pressure of the filtered air, heating element for heating the pressure regulated air, a blower to push the pressurized air at a required flow rate; and a counter-flow tube for directing the heated air to a face mask wherein the air purifier mask is disinfected periodically, using the heating elements.

Provided is a method which performs filtering incoming air, at an inlet of an air purifying mask, regulating pressure of the filtered air using the pressure sensors, moving the pressure regulated air at a required flow rate using a blower, heating the pressure regulated air using heating elements, directing the heated air to a face mask, using a counter flow tube and disinfecting the air purifying mask periodically using the heating element.

### BRIEF DESCRIPTION OF THE DRAWINGS

FIG. 1 describes an embodiment of the air purifier mask;

FIG. 2A describes the components of an embodiment of the air purifier mask;

FIG. 2B describes an embodiment of the components in the casing of the air purifier mask;

FIG. 3 describes an embodiment of the high level controller architecture of the air purifier mask; and

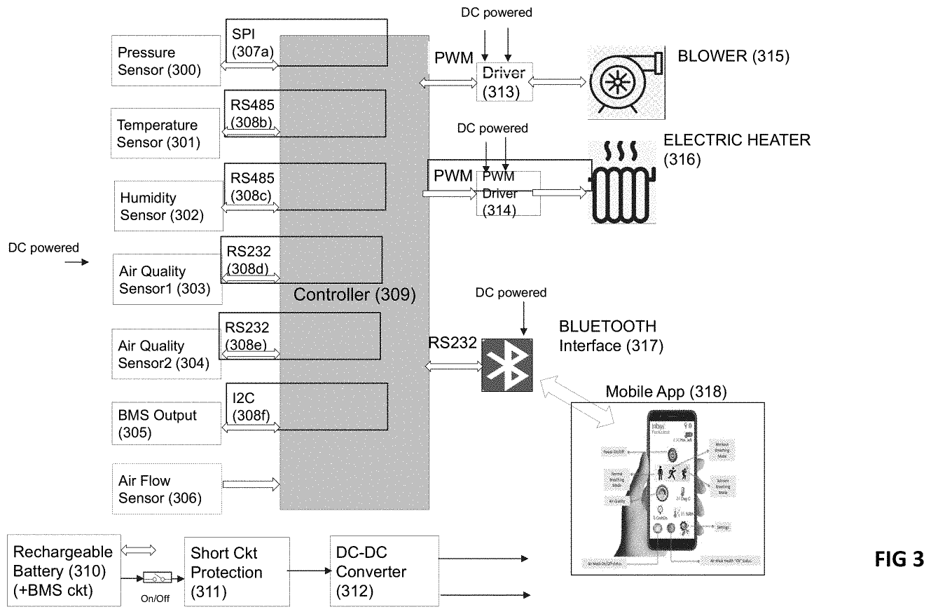

FIG. 4 describes an embodiment of the process of implementing the air purifier mask.

### DETAILED DESCRIPTION

The present disclosure provides a strategic air purifying respirator or a mask, enabling a pure, comfortable, and effortless respiration, all-day, for home, office, hospital & polluted environments. It helps prevent the spread of infection and prevent the individual from contracting any airborne infectious germs.

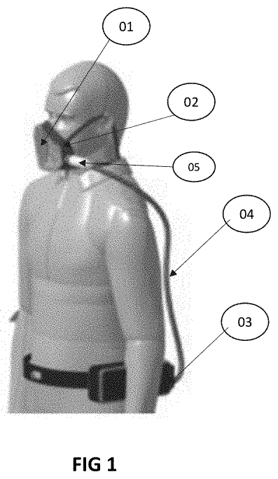

An embodiment of the air purifying mask as per the present disclosure will be explained along with FIG. 1. The disclosure describes a face mask (01) along with its components. Some of the components maybe housed inside a casing (03). The casing may contain a blower, an air filtering system, along with other associated components for the mask including appropriate pressure, flow control and filtration settings. An air quality checking capability maybe provided with the air mask. The air quality checking may comprise checking by some sensors, air particles or micro dust particles in air which may not be good for inhaling. A flexible attachable/detachable connecting coaxial duct for inhale and exhale, may connect the face mask and the casing having the air filtration system. The face mask and the casing may also be connected by any other tube, with a part of it connected at the face mask being a coaxial or concentric tube. The system may have a heating element for self-cleaning and temperature control. The heating element may also help for disinfection of air path within the system & air mask.

In an embodiment this air mask maybe a polycarbonate face mask and may be provided with a seal lining (02). The seal lining may be silicone or any other similar material. The mask maybe designed for everyday use, in all terrain, and all weather. The usage of the mask may not be limited to infectious or affected surrounding air.

In an embodiment, the air mask may provide micro ventilation through controlled & filtered air supply. This may also enable induced breathing with high pressure air supply. These maybe achieved by a flow control valve at the outlet, and a wireless pressure sensor installed in the face mask. The flow control valve may help release the exhaled air when the pressure in the mask reaches a predetermined and calculated value. The pressure in the face mask can be monitored by the wireless pressure sensor. This may be used in case of high altitude, low atmospheric pressure, or respiratory ailments.

These may be powered from a portable battery system. This may also help in removing stale or humid air due to exhalation from the mask. This system may also provide both air quality and quantity control.

In an embodiment, the air mask may provide filtered air and breathing assistance to a user in various modes that may include exercise mode, normal breathing mode, ailment mode and similar modes.

In an embodiment, the air mask may notify the performance of the electrical, electronic, and mechanical modules available with it from the feedback control loop. The air mask may notify the user with an alarm, when air supply or any module is faulty, or performance is low. The air purifier mask can be used either in powered or non-powered mode. It has dual purpose usage concept. A user has the option to use the mask in non-powered mode as well. During this mode, the casing may not be needed to be carried e.g., when travelling in a crowded place.

In one embodiment, the implementing architecture of the air mask may include various components which can help set the air pressure and temperature as well. Maximum airway pressure can be adjusted as required, by adjusting the flow rate from the blower. This may enable for quick reaction evacuation, e.g. against sudden sneezing/coughing.

A rechargeable battery can also be provided along with the air mask as a power source for functioning and controlling of the system.

In one embodiment, this air mask can be controlled through an external interface device, such as mobile app. The user can configure the interface to display and set the various parameters such as, air temperature, humidity and flow rate. The air mask system can continuously monitor the battery capacity and may also display the battery status in the interface device. Through the interface, user can set breathing mode related parameters for controlled air flow. The parameter values can also be stored and retrieved for future reference as and when needed.

The air mask may be connected to the casing through a single flexible hose (04), with air inlet and outlet within a coaxial tube arrangement. The mask outlet may have with filters, either within mask or at the end of the outlet duct.

In one embodiment, the air purifier mask maybe configured to provide a desired respiratory rate. For the purpose of example, the average respiratory rate may be a range of between to 10-30 breathes per minutes in increments of 2. The respiratory rate provided by the air mask may change as per the mode chosen by a user.

In an embodiment, the air purifier maybe configured to provide an optimal tidal volume. The tidal volume maybe defined as amount of air moving into lung in one cycle. For the purpose of example, the tidal volume for an average person may be 300-400 ml per breath. It could typically be 6 ml/Kg Weight of patient. 70 Kg Person may require 420 ml per breath and could have a tidal volume range of 250-600 ml in steps of 50m1. The flow rate is provided by the modes chosen by the user.

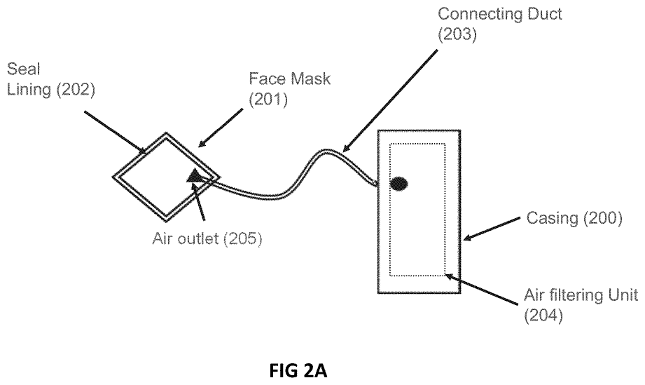

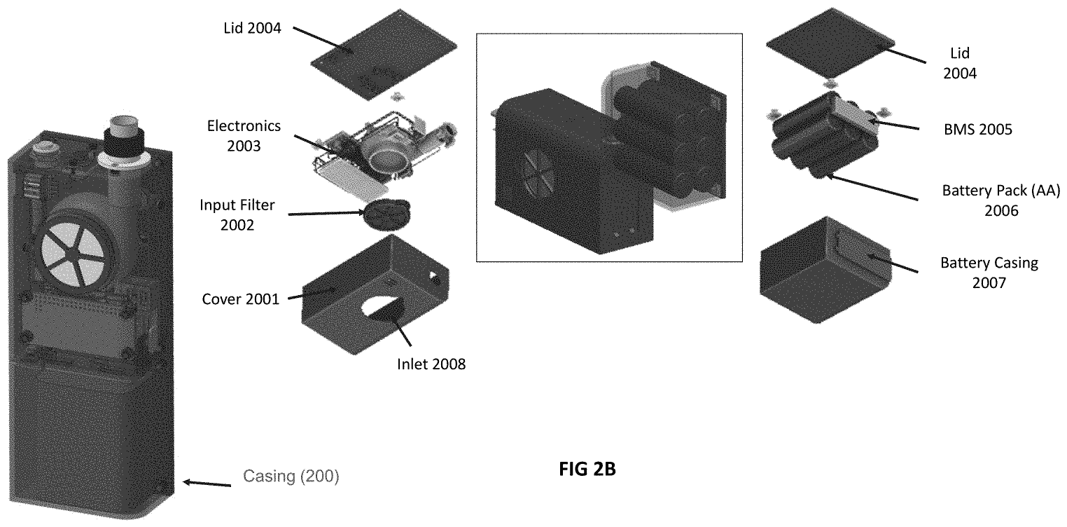

A detailed explanation of an embodiment of all the components of the air purifier is provided along with the description of FIG. 2A.

In an embodiment, the air purifier mask comprises a face mask (201). The face mask may have a seal lining (202) to make it fit properly, and not let the exhaled air fog the users glasses etc. The seal lining may comprise of silicon.

In one embodiment, in the unpowered mode, the face mask may be used with a replaceable filter without the casing. The filter in the unpowered face mask may have a filter guard, and the filter guard may be a snap fit. In the powered mode, with the casing, there may be no filter on the face mask. In the powered mode a prefiltered and treated air reaches the face mask. Hence the face mask may not need a separate filter in a powered mode.

In one embodiment, the face mask may have a stretchable and adjustable head strap for secure and comfort fit.

In one embodiment, the face mask may be connected to a casing (200) through a flexible attachable/detachable connecting duct (203). One end of the connecting duct near the face mask, may be a counter flow tube, implemented using a connecting coaxial duct. In an embodiment, the coaxial duct can be a counter flow tube or a concentric tube. Any other appropriate connecting duct providing concentric air flow can be used to connect the air mask to the casing.

In one embodiment, the face mask may have an air outlet (205). The exhaled air may be pushed out through the outlet. In an embodiment, a part of the connecting duct near the face mask maybe a concentric tube. The concentric tube arrangement may be used for filtering the exhaled air. The outer of the concentric tube may have a filter in it. The exhaled air before being released to atmosphere gets filtered. The outlet filter may be a N95 filter or any other appropriate filter. Filtered and treated air may enter the face mask through the inner pipe of the concentric pipes.

Exhaled air may move out through the annular area of the concentric pipes.

The casing may contain an air filtering unit (204). The air filtering unit may comprise of multiple components which treat the incoming air before a user inhales it. The components in the casing (200) will now be described along with FIG. 2B. The casing can be carried as a wearable backpack, a sling bag (hang on shoulders) or around the waist. It can be carried or worn in any other convenient way. The casing may house multiple components for configuring various parameters related to the input air flow. In an embodiment, the casing may comprise an input filter (2002) at an inlet (2008) for the air. A user may use a desired micron particle filter, as per the requirements. For the purpose of illustrations only, a user may choose to use a PM2.5 filter. In another example, less than 95 microns particle are filtered out at the inlet. The input filter may comprise a filter cartridge, and maybe attached to a cover part (2001) of the casing. Any other filtering component capable of removing particles beyond a decided dimension can be used. In one embodiment the filter cartridge maybe replaceable.

In an embodiment, the input filter maybe connected to the electronics (2003) which may enclose multiple components. In an embodiment it may comprise, but not limiting to, a blower, a pressure sensor, a pressure regulator, a temperature and humidity sensor, heating element. The components including multiple sensors and other required components may be arranged on a PCB. The pressure sensor may monitor the pressure of the filtered air. The air pressure may be adjusted using a pressure regulator. The blower may adjust the flow of the pressure regulated air. It pushes the air forward. In an embodiment, the blower may be attached near the inlet before the pressure sensor. Filtered air passes over the pressure sensor. The air pressure may be monitored and adjusted automatically or may also be adjusted manually. This enables pressurized air for comfortable semi-induced breathing. In one embodiment, the pressure sensor can be fully calibrated, and temperature compensated for offset, sensitivity, temperature, and non-linearity. Any appropriate pressure sensor may be used that satisfy the prefect repeatability, linearity, stability, and sensibility.

In an embodiment, the electronics (2003) further comprise of a temperature & humidity sensor. It may include a resistive-type humidity measurement component, an NTC temperature measurement component and a high-performance 8-bit microcontroller inside and provides calibrated digital signal output. Other configuration and categories of these measurement components can also be used.

In another embodiment, an appropriate heating element is used. The heating element maybe embedded in a set of tubes within the electronics (2003). The tubes can be any appropriate dimension and type. The pressurized air may pass through the temperature & humidity sensor, and then the heating element in the set of tubes. The heating element may heat the pressurized air to a desired temperature. This may be automatically set or can be manually adjusted for conditioning the air as appropriate. Humidity sensor may help to detect water content in the pressurized air. The air temperature may be adjusted to remove or adjust moisture from the air.

In an embodiment, the heating element may also be used for regulating the temperature of the filtered air, as per the weather.

In an embodiment, the heating element maybe configured for disinfecting the air passage route. The route may comprise the connecting tube, and the face mask. The air passage route may also comprise other appropriate components. This may be manually done by the user as and when needed, or the user may set a frequency and timer for disinfecting the air purifier mask. In another embodiment, the air purifier mask may be attached to a charging point for the chargeable batteries. The heating elements may be switched on during the charging time of the air purifier. The temperature of air rises to disinfect the mask.

In one embodiment, the disinfection may be done when the air purifier mask is not being used. The heating element may generate heat and disinfect the components and kill any type of infection. This may also be initiated by the user through the remote software. The heating element provides an embedded auto disinfection system.

The above component of the electronics (2003) maybe covered by a lid (2004). This may help the user avoid coming in touch with the electronic component.

In an embodiment, the electronics (2003) maybe connected to a power source. The power source maybe chargeable or non-chargeable or replaceable batteries (2006). These batteries and the required power may be controlled by a Battery Management System (BMS) (2005). In an example, an 8000 mAh battery may provide 8 hours of performance. This may also depend on the operating modes, e.g. full power mode, or normal use mode or similar such modes. These batteries maybe packed in a battery casing (2007). This may be connected to the electronics (2003). In an embodiment the battery casing and the electronics are together included in the casing (200) for the air purifier mask.

In one embodiment, the casing and the electronics comprised in it, may not be used. A user can use only the face mask without the electronics. The mask may then perform as a conventional air mask. This is the unpowered mode. The face mask in this mode, may comprise of a filter and a filter cover. In this mode, the user need not carry the casing.

In one embodiment, the air purifier mask maybe connected to a remote software. The remote software maybe a mobile application. The air purifier mask maybe connected to the mobile application through Bluetooth or internet or any other applicable connection technology.

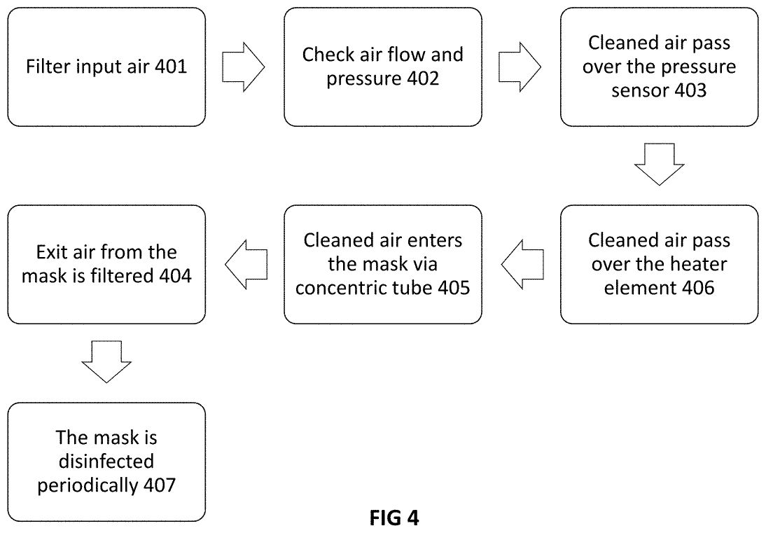

An embodiment of the high-level architecture of the present disclosure will now be explained along with the description of FIG. 4. In an embodiment, multiple sensors in the casing of the air purifier may send their readings to a mobile application, through a remote connection. In one example the remote connection can be Bluetooth.

In an embodiment, the sensors in the casing maybe powered by a DC source. The sensors used maybe, but not limited to, Pressure sensor (300), Temperature Sensor (301), Humidity Sensor (302), Air Quality Sensor1 (303), Air Quality Sensor2 (304), BMS Output (305), Air Flow Sensor (306). The pressure sensor monitors the pressure of the incoming filtered air, which can accordingly be regulated, as mentioned in the earlier paragraphs. The temperature sensor monitors the temperature of the pressurized air. The temperature can accordingly be regulated as mentioned in the earlier paragraphs. The humidity sensor monitors the humidity or the hydration in the air. In an embodiment, the humidity or hydration of the pressurized air may also be controlled by the heating element. In one embodiment, the sensor may include a resistive-type humidity measurement component, an NTC temperature measurement component and a high-performance 8-bit microcontroller which provides calibrated digital signal output. A blower (315) may also be attached to adjust the flow of air towards the sensors.

In one embodiment, the electronics (2003) may also comprise one or more air quality sensors (303, 304). The air quality sensor maybe used to sense the dust particles in the inhaled air.

In one embodiment, the casing may comprise a Battery Management System (BMS) output (305). This component may check and regulate the power supply. It may also indicate in case of weaker output received from BMS, which may be an indication of battery replacement, or any other corrections or update required.

In one embodiment, the air flow sensor maybe used to measure mass flow of the air in the purifier. In an example, a specified air flow rate from 12-80 l/min maybe needed for different operating modes.

In one embodiment, the above described components may be connected to a Controller (309), through various pins and chips, appropriate. Some of the chips and IC that maybe used are, but not limiting to, SPI (307a), RS485 (308b), RS232 (308d), RS232 (308e), or I2C (308f). A user may use any other connecting options as appropriate based on the Controller (309), or the sensor architectures and other related requirements.

In one embodiment, the architecture of the purifier may further comprise battery circuit (BMS circuit) (310). This may provide output to BMS output (305). The battery circuit may alternatively include a rechargeable battery circuit. This may be connected to an ON/OFF switch, and a short circuit protection circuit (311).

This may provide protection to the electronics from any accidental damage, or any other issues related to wiring etc.

In one embodiment, the short circuit protection circuit (311) maybe connected to a DC-DC converter (213) which helps to protect the circuit.

In an embodiment, the various electronics as explained in an earlier paragraph, which may include a heating element, the pressure regulator, a blower (315), and other components may be connected to the controller (309), through appropriate drivers (313, 314). The various drivers may also be DC powered from the BMS circuit (310).

In an embodiment, the controller (309) maybe connected to a remote software through a connection interface. The connection interface (317) maybe, but not limited to, Bluetooth. The interface may also be DC powered from the BMS circuit (310). In one embodiment, the remote software maybe a mobile application (318). The remote software may also be a desktop software.

In an embodiment, the electronic components are remotely controlled by the software. The software may help power on/off the purifier. The various sensors and drivers as described in above figures may be controlled by the software. A user may operate his air purifier mask using the remote software.

In an embodiment, when a user initiates the remote software, the controller (309) may get initialized. The Bluetooth connectivity of the air purifier mask, with the remote software maybe checked. Once the connection is established or verified, the user commands are received. A user may give commands to set a blower speed for the air flow, set the heater value and other required values for the various drivers and the parameters setting.

In one embodiment, the interface of the remote software enables a user to select an appropriate mode for the working of the air purifier mask. The modes can be one of, but not limited to, workout breathing mode, normal mode, and ailment breathing mode. These modes may have a fixed setting for the heating element, pressure regulator and other such components. A user may choose the mode in which he may want the air purifier mask to work.

In an embodiment, the remote software on a mobile device provides a display to user which may show the readings of the various sensors, along with the air quality reading. The sensor readings can also be stored and retrieved for future references as and when needed.

In an embodiment, the air purifier mask may notify the performance of the electrical, electronic, and mechanical modules available with it from the feedback control loop. The mask may notify the user with an alarm, when air supply or any module is faulty, or performance is low.

The working of the air purifier mask will now be explained in detail using the description of FIG. 4. Initially the air entering the mask through the inlet in the casing, passes through a filter (401). The air is purified through the filter. The filter may be used as per the user requirement, and a specific dimension filter can be used. The purified air is then checked and regulated for air flow and air pressure (402, 403).

The regulation of the air pressure and the air blow can be done as per the mode selected by a user using the remote software. It can be also be set manually by the user. The settings for each of the modes can also be configured by the user.

In an embodiment, the pressurized air then passes through a heating element (406). This may also include a temperature sensor, for checking the temperature of the air. The temperature till which the air has to be heated may be adjusted and controlled using the remote software, and the modes as described earlier.

In an embodiment, the clean and treated air is directed to the face mask (405). The mask is connected to the casing through a set of detachable tubes. In one example a part of these tubes may be concentric where it connects with the mask.

Once the treated air reaches the mask, the user inhales it. The mask may have a sealing lining to avoid air or humidity from leaking or fogging the user glasses. In an example the sealing lining may be silicon lining.

In an embodiment, the exhaled air maybe filtered using the concentric tubes (404). This may ensure that infected air exhaled by the user is not released into the atmosphere. The exhaled air passing through the outer tube in the concentric tube may get filtered, before going to atmosphere. The outer tube may have appropriate filters. Various embodiments of the implementation of the process as disclosed here, have been elaborated in detail in the earlier paragraphs.

In one embodiment, the mask may be disinfected periodically using the heating element as described in earlier paragraphs.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. An air purifier mask comprising,
a face mask;
a casing connected to the face mask, the casing comprising,
an inlet filter for filtering incoming air;
one or more pressure sensors and regulators for monitoring and regulating a pressure of the filtered air;
one or more heating element for heating the pressure regulated air and periodically disinfecting the air purifier mask;
a blower to push the pressurized air at a required flow rate; and
a counter-flow tube for directing the heated air to the face mask.

2. The air purifier mask as claimed in claim 1, wherein the incoming air is filtered at an inlet of the air purifier mask using the inlet filter configured to filter out particles of preset dimensions.

3. The air purifier mask as claimed in claim 1, wherein exhaled air is flown out using the counter-flow tube.

4. The air purifier mask as claimed in claim 3, wherein the exhaled air is filtered before being released through an outlet.

5. The air purifier mask as claimed in claim 1, wherein the air purifier mask is controlled and monitored by a mobile application.

6. The air purifier mask as claimed in claim 5, wherein the mobile application is configured to operate at modes comprising normal mode, workout mode and ailment mode.

7. The air purifier mask as claimed in claim 1, wherein the filter, the one or more pressure regulator and the one or more heating element are provided in the casing.

8. The air purifier mask as claimed in claim 1, wherein the air purifier mask is powered using a power source comprising a chargeable battery or a non-chargeable battery.

9. The air purifier mask as claimed in claim 1, wherein the face mask is used in an unpowered mode, the face mask comprising an air filter in the unpowered mode.

10. A method comprising,
filtering incoming air, at an inlet of an air purifying mask;
regulating pressure of the filtered air, using one or more pressure sensors;
moving the pressure regulated air at a required flow rate using a blower;
heating the pressure regulated air, using one or more heating elements;
directing the heated air to a face mask, using a counter flow tube; and
disinfecting the air purifying mask periodically using the heating element.

11. The method as claimed in claim 10, comprising filtering the input air at an inlet of the air purifying mask using an input filter configured to filter out particles of preset dimensions.

12. The method as claimed in claim 10, comprising transferring out exhaled air using a counter-flow tube in the connecting tube.

13. The method as claimed in claim 12, wherein the exhaled air is filtered before being released through an outlet.

14. The method as claimed in claim 10, comprising controlling and monitoring the air purifier mask by a remote software.

15. The method as claimed in claim 14, wherein the remote software is configured to operate at modes comprising normal mode, workout mode and ailment mode.

16. The method as claimed in claim 10, wherein the filter, the one or more pressure regulator and the one or more heating element are provided in a casing.

17. The method as claimed in claim 10, wherein the air purifier mask is powered using a power source comprising a chargeable battery or a non-chargeable battery.

18. The method as claimed in claim 10, wherein the face mask is used in an unpowered mode, the face mask comprising an air filter in the unpowered mode.
